(19) United States
(12) Patent Application Publication
(10) Pub. No.: US 2024/0268729 A1
(43) Pub. Date: Aug. 15, 2024

(54) Finger Cuff Device With Lancet Guide and Integrated Absorbent Pad for Capillary Sample Collection

(71) Applicant: Becton, Dickinson and Company, Franklin Lakes, NJ (US)

(72) Inventors: David Colie Wight, West Orange, NJ (US); Shridhar Shankar Palekar, Lyndhurst, NJ (US)

(21) Appl. No.: 18/567,392

(22) PCT Filed: Jun. 3, 2022
(86) PCT No.: PCT/US22/32115

§ 371 Date: Dec. 6, 2023

Related U.S. Application Data
(60) Provisional application No. 63/197,784, filed on Jun. 7, 2021.

(51) Int. Cl. A61B 5/15; A61B 5/151

(52) U.S. Cl. CPC A61B 5/150748; A61B 5/150343; A61B 5/15105; A61B 5/15142

(57) ABSTRACT

A finger cuff device for use in capillary blood sample collection, including a finger cuff having an open end portion sized to accommodate a user's finger, a finger stop end portion opposite the open end portion, an outer sidewall, and an inner sidewall. The finger cuff also includes at least one lancet guide opening formed through the outer sidewall and inner sidewall. Additionally, the finger cuff device includes at least one integrated absorbent pad positioned on the inner sidewall of the finger cuff.

### CROSS-REFERENCE TO RELATED APPLICATION

The present application claims priority to U.S. Provisional Application Ser. No. 63/197,784, entitled “Finger Cuff with Lancet Guide and Integrated Absorbent Pad for Capillary Sample Collection”, filed Jun. 7, 2021, the entire disclosure of which is hereby incorporated by reference in its' entirety.

### BACKGROUND OF THE INVENTION

### Field of the Invention

The present disclosure relates to a wearable finger cuff device to aid in capillary blood sample collection. More specifically, the present disclosure relates to a patient-wearable finger cuff having a lancet guide opening and integrated absorbent pad.

### Description of Related Art

Capillary blood draw is often preferable to venous blood draw, particularly in a non-clinical setting and/or small blood volume applications. Capillary collection relies upon a lancet to make a small puncture in, e.g., a patient's fingertip, which draws a smaller sample volume as compared to venous collection, and is generally more comfortable for the patient. Additionally, the lack of a hypodermic needle results in lower risk and less anxiety for the patient. In fact, because the process for capillary collection is relatively simple, patients themselves can be taught to collect a sample, making blood tests more accessible and less expensive.

However, when sample collection performed by the patient or another person without medical training, errors can be made during capillary blood draw which may negatively impact patient experience and sample quality. One such error is the puncture of the patient's finger in a less-than-ideal location. Generally, it is recommended that the finger puncture be performed at a slightly off-center location on the pad of the finger. If the puncture is executed outside of this preferred location, the result may be low blood flow from the puncture and/or pain for the patient.

Another possible error made by the patient or untrained assistant during capillary collection may be that the first drop of blood drawn from the finger is into the sample tube. Typically, it is recommended that the first drop of blood be wiped away with separate gauze (and, thus, not collected), as this first drop may contain contaminants that can impact the integrity of sample. Additionally, the first drop of blood may also contain platelet tissue factor, which can undesirably begin the blood clotting process and may prevent analysis from being performed.

### SUMMARY OF THE INVENTION

Accordingly, a finger cuff device for use by a patient or untrained person during capillary blood collection, wherein the finger cuff device includes both a lancet guide opening located at an optimal puncture location and an integrated absorbent pad so as to ensure removal of the first drop of blood upon removal of the finger cuff device.

In accordance with an embodiment of the present disclosure, a finger cuff device for use in capillary blood sample collection includes a finger cuff having an open end portion sized to accommodate a user's finger, a finger stop end portion opposite the open end portion, an outer sidewall, an inner sidewall, and at least one lancet guide opening formed through the outer sidewall and inner sidewall. The finger cuff device also includes at least one integrated absorbent pad positioned on the inner sidewall of the finger cuff.

In certain configurations, the at least one lancet guide opening is positioned off-center from a centerline of the finger cuff. In other configurations, the at least one lancet guide opening is positioned proximate the finger stop end portion of the finger cuff. In still other configurations, the at least one lancet guide opening is elongated relative to a longitudinal axis of the finger cuff. Optionally, the at least one integrated absorbent pad is positioned on the inner sidewall in line with the at least one lancet guide.

In another configuration, the device includes a pair of open portions formed on respective sides of the finger cuff. Optionally, each open portion further comprises a rounded end portion. In certain arrangements, the finger cuff is formed of an elastic material.

In accordance with another embodiment of the present disclosure, a kit for capillary blood sample collection includes a lancet, a microtainer collection tube, at least one sterile wipe, a bandage, and a sterile finger cuff device. The sterile finger cuff device includes an open end portion sized to accommodate a user's finger, a finger stop end portion opposite the open end portion, an outer sidewall, an inner sidewall, at least one lancet guide opening formed through the outer sidewall and inner sidewall, and at least one integrated absorbent pad positioned on the inner sidewall of the finger cuff.

In certain configurations, the at least one lancet guide opening of the finger cuff is positioned off-center from a centerline of the finger cuff. The at least one lancet guide opening may be positioned proximate the finger stop end portion of the finger cuff. Optionally, the at least one lancet guide opening of the finger cuff is elongated relative to a longitudinal axis of the finger cuff. The at least one integrated absorbent pad of the sterile finger cuff device may be positioned on the inner sidewall in line with the at least one lancet guide. In one configuration, the sterile finger cuff device further includes a pair of open portions formed on respective sides of the finger cuff. Optionally, each open portion of the finger cuff further includes a rounded end portion. The finger cuff may be formed of an elastic material.

In accordance with another embodiment of the present invention, a method of capillary blood sample collection includes preparing a finger and desired puncture site for blood sample collection with a sterilized wipe, placing a sterile finger cuff device over the prepared finger at the desired puncture site, the sterile finger cuff device including an open end portion sized to accommodate a user's finger, a finger stop end portion opposite the open end portion, an outer sidewall, an inner sidewall, and at least one lancet guide opening formed through the outer sidewall and inner sidewall. The method also includes at least one integrated absorbent pad positioned on the inner sidewall of the finger cuff, pressing a lancet through the at least one lancet guide opening to puncture the finger at the desired puncture site, and removing the sterile finger cuff device from the finger to expose the puncture site and wipe away a first blood drop from the puncture site via the at least one integrated absorbent pad of the sterile finger cuff device.

In certain configurations, the method also includes collecting a blood sample in a microtainer tube after the first blood drop is wiped away from the puncture site via the at least one integrated absorbent pad of the sterile finger cuff device. Optionally, the method further includes closing the microtainer tube after collection of a desired volume of blood sample and bandaging the puncture site on the finger. In certain configurations, the method also includes placing the sterile finger cuff device over the prepared finger comprises placing the finger cuff on the finger such that the at least one lancet guide opening is positioned off-center of a centerline of the finger.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is an isometric view of a finger cuff device in accordance with an aspect of the present disclosure;

FIG. 2 is a side cross-sectional view of the finger cuff device of FIG. 1;

FIG. 3A is an isometric view of the finger cuff device in a pre-collection condition;

FIG. 3B is an isometric view of the finger cuff device in a collection condition;

FIG. 3C is an isometric view of the finger cuff device in a post-collection condition;

FIG. 4 is a flowchart of a method of capillary blood collection in accordance with an aspect of the present disclosure.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

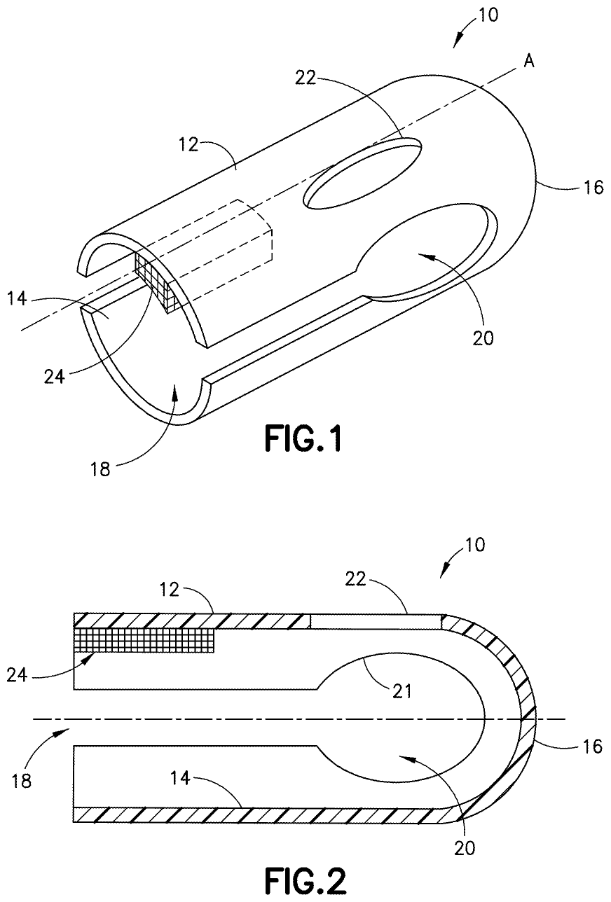

Referring to FIGS. 1 and 2, a finger cuff device 10 in accordance with an aspect of the present disclosure is illustrated. Finger cuff device 10 includes an outer sidewall 12, an inner sidewall 14, a finger stop end portion 16, and an open end portion 18. Additionally, a pair of open portions 20 are formed on respective sides of the finger cuff device 10. Each open portion 20 includes a rounded end portion 21, which enables the finger cuff device 10 to expand relative to the open end portion 18 so as to accommodate a user's finger.

In the embodiment shown in FIGS. 1 and 2, the finger stop end portion 16 is substantially closed, which prevents the finger cuff device 10 from traveling beyond a desired position relative to the user's fingertip. However, it is to be understood that finger stop end portion 16 need not be fully or substantially closed, and may include one or more openings formed therethrough. For example, in some embodiments, the finger stop end portion 16 may comprise one or more narrow strips of material that function to provide a limit or stop for the finger cuff device 10.

In some embodiments, the finger cuff device 10 is formed of an elastic material, thereby allowing the finger cuff device 10 to deflect when initially positioned on a user's finger, yet return toward an original position so as to better hold the finger cuff device 10 in place on the user's finger without additional securement via, e.g., another hand of the user, an adhesive such as medical tape, etc. The finger cuff device 10 may be formed of any appropriate elastic material capable of sterilization, such as, e.g., plastic, polymer, metal, etc. However, it is to be understood that other materials, including non-elastic materials, may be utilized.

Referring still to FIGS. 1 and 2, the finger cuff device 10 further includes at least one lancet guide opening 22 formed at a position proximate the finger stop end portion 16. As is shown in FIG. 1, the lancet guide opening 22 is positioned slightly off-center from a finger centerline A of the finger cuff device 10. In this way, the lancet guide opening 22 provides a guide or template for an ideal puncture location via, e.g., a lancet (not shown). As noted above, the preferred location for capillary blood draw is from an off-center location on the user's finger pad. Thus, lancet guide opening 22 provides a guide for identifying such a location(s), while also blocking undesirable locations such as the center of the finger pad, the tip of the finger, etc. Additionally, as the finger stop end portion 16 limits the travel of the finger cuff device 10 relative to the fingertip, the lancet guide opening 22 is positioned such that the puncture location is sufficiently away from the joint of the user's finger.

The lancet guide opening 22 is sized to sufficiently accommodate at least a piercing tip of a lancet. In some embodiments, lancet guide opening 22 is elongated so as to enable the user to choose from several off-center puncture locations. An elongated lancet guide opening 22 may be beneficial for a user performing somewhat frequent blood collection procedures, as it may enable the user to avoid re-puncturing the same location with each collection event.

Furthermore, while not shown in FIGS. 1 and 2, it is to be understood that at least one additional lancet guide opening 22 may be provided on an opposite side of the finger centerline A. In this way, the user may choose a preferred puncture location on an off-center finger pad location and/or alternate which side of the finger they choose as the puncture site.

Additionally and/or alternatively, in some embodiments, at or near the lancet guide opening 22, the finger cuff 10 could incorporate a locking mechanism (not shown) capable of capturing the lancet after the finger puncture is performed. The locking mechanism would be configured to prevent removal of the lancet separate from removal of the finger cuff 10 in its entirety. Such a configuration would eliminate the need to separately dispose of the lancet and the finger cuff 10, minimizing the number of actions the user (or another person assisting the user) would need to take prior to collecting the capillary blood sample. Furthermore, due to the presence of the locked lancet, the user would be forced to fully remove the finger cuff 10 in order to access the puncture site, preventing attempted blood draw with the finger cuff 10 still in place on the user's finger.

Referring still to FIGS. 1 and 2, finger cuff 10 further includes an integrated, sterile absorbent pad 24. In some embodiments, the absorbent pad 24 is located on the interior sidewall 14, proximate the open end portion 18 and substantially in-line with the lancet guide opening 22. The absorbent pad 24 may be affixed to the interior sidewall 14 by any appropriate means and/or method such as, e.g., an adhesive. Furthermore, the absorbent pad 24 may be formed of any appropriate absorbent material such as, e.g., woven or non-woven cotton gauze, polyester gauze, absorbent paper, etc.

As noted above, during typical capillary blood collection, it is preferable for the first blood drop to be wiped away by the user via a separate gauze pad prior to blood collection. This additional step adds complexity to the procedure, and may even be overlooked entirely, particularly when the collection procedure is being performed by the patient themselves or another inexperienced user. However, integrated absorbent pad 24 of finger cuff 10 prevents such an oversight, as removal of the finger cuff 10 after puncture necessarily causes the absorbent pad 24 to pass over the puncture site, thereby removing the first blood drop without the need for an additional wiping step. Additionally, the location of the absorbent pad 24 helps to minimize discomfort during removal of the finger cuff 10, as it substantially protects the puncture site from contact with other surfaces of the finger cuff 10 during removal.

While not shown, it is to be understood that more than one absorbent pad 24 may be provided on interior sidewall 14 and/or on other locations of finger cuff 10. For example, if more than one lancet guide opening 22 is provided in finger cuff 10, an associated absorbent pad 24 may be provided relative to each lancet guide opening 22. Additionally and/or alternatively, the size of absorbent pad 24 is not limited to that which is shown in FIGS. 1 and 2. For example, the absorbent pad 24 may be larger or smaller than that which is shown in FIGS. 1 and 2.

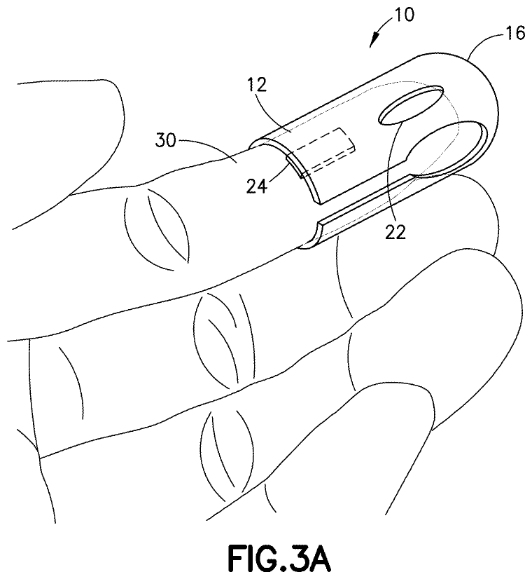

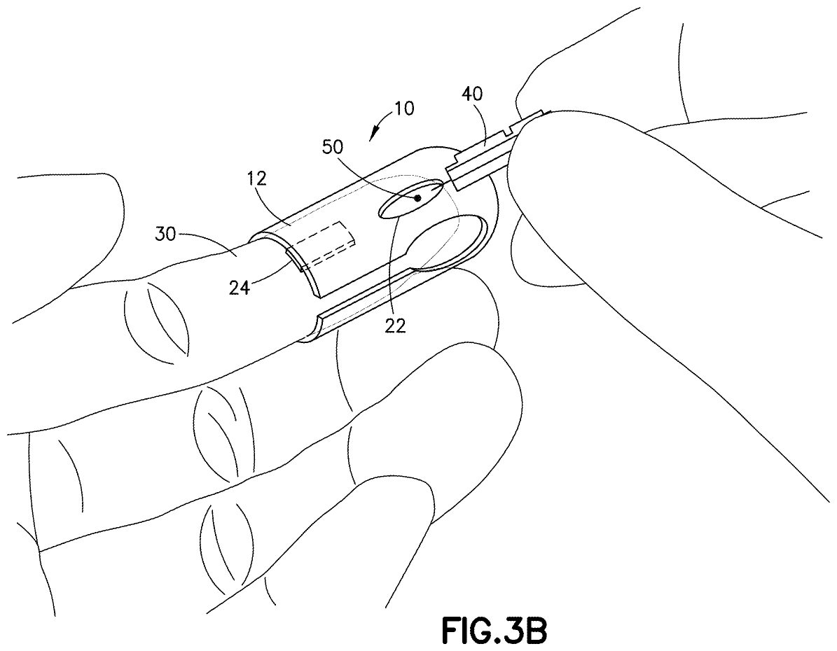

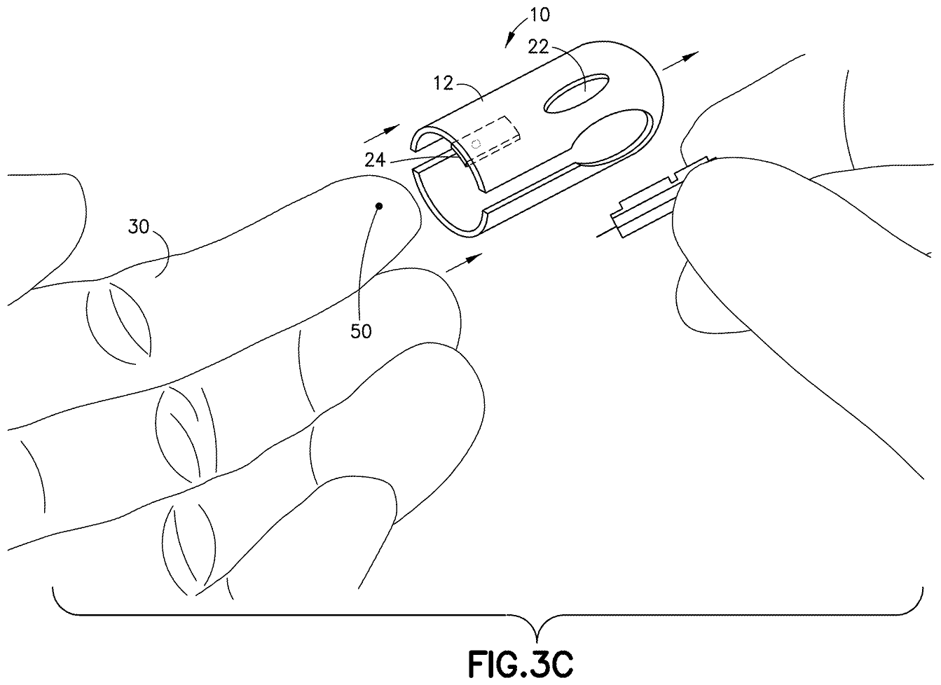

Referring now to FIGS. 3A-3C, use of finger cuff 10 on a user's finger 30 in accordance with an aspect of the present disclosure is illustrated. As shown in FIG. 3A, the user first places the finger cuff 10 over the tip of a selected finger 30 such that the lancet guide opening 22 reveals an off-center portion of the pad of finger 30, demarcating a desired puncture site. As detailed above, the finger cuff 10 is preferably held on the finger 30 via the elasticity of the finger cuff 10 itself. However, other means of securement of the finger cuff 10 may also be utilized, such as, e.g., an adhesive.

Referring to FIG. 3B, after the finger cuff 10 is placed, the user (or another person) utilizes a lancet 40 in order to puncture the user's finger 30 at a puncture site 50 within the lancet guide opening 22. Then, as shown in FIG. 3C, after confirming that a puncture site 50 did successfully draw blood, the user (or another person) removes the finger cuff 10 from the finger 30 in a distal direction away from the user's fingertip. This removal of the finger cuff 10 simultaneously passes the absorbent pad 24 over the puncture site 50, thereby wiping the first drop of blood away from the puncture site 50, which allows the blood sample to be preserved for a longer time period before testing and/or reduces the likelihood of contamination. The user need not perform a separate wiping step, and collection of the capillary blood sample in, e.g., a microtainer tube may begin immediately after removal of the finger cuff 10.

As detailed above, the finger cuff 10 simplifies capillary blood sample collection by the patient themselves and/or another unskilled person, ensuring that an appropriate puncture site is selected and that the first blood drop is wiped away prior to collection. As the finger cuff 10 incorporates the lancet guide opening 22 and absorbent pad 24 in a single device, single-handed collection by the user themselves is also made possible. Moreover, as the finger cuff 10 merely facilitates a proper sample collection technique and does not itself impact sample quality, few risks to the patient and/or the sample are possible.

In some embodiments, the finger cuff 10 may be provided as part of an overall sample collection provided to the user for at-home capillary blood sample collection. The kit may include, e.g., a lancet, a microtainer collection tube, at least one sterile wipe, the sterile finger cuff 10, and a bandage for use on the puncture site post-collection.

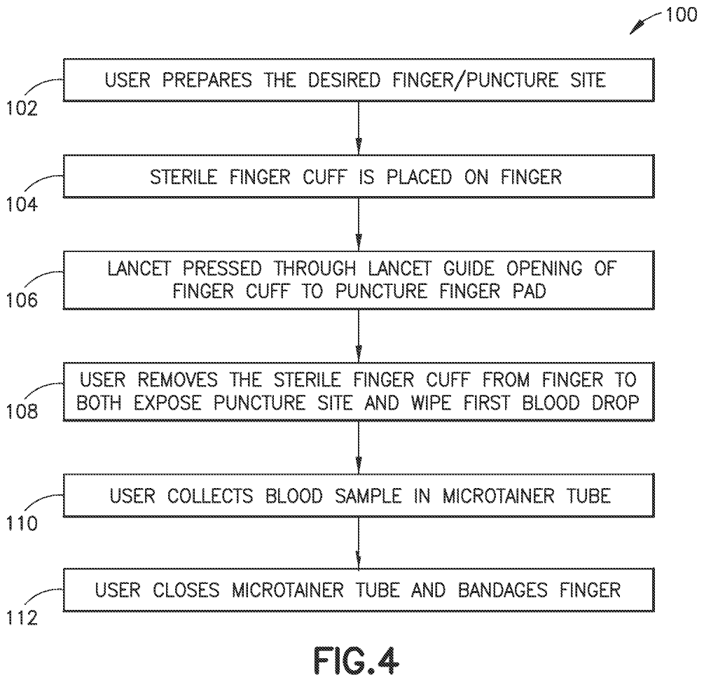

Next, referring to FIG. 4, a method 100 of capillary blood sample collection in accordance with an aspect of the present disclosure is illustrated. First, at step 102, the user (or another person) selects a desired finger/puncture site and prepares the site for collection of a blood sample. The preparation may include, e.g., cleaning of the puncture site with a sterile wipe. Next, at step 104, a sterile finger cuff such as that described above with respect to FIGS. 1-3C is placed on the selected finger. At step 106, a lancet is pressed through the lancet guide opening of the finger cuff in order to puncture the user's finger pad at the selected puncture site, thereby drawing a first drop of blood from the puncture site. The lancet may be held by the user themselves or by another person.

Next, at step 108, the user (or other person) removes the finger cuff from the finger, which simultaneously exposes the puncture site and wipes the first blood drop from the puncture site, as described above with respect to FIGS. 1-3C. Upon removal of the finger cuff, the user may dispose of the finger cuff and/or lancet in an appropriate container.

After appropriate removal of the finger cuff and the first blood drop, at step 110, the user (or other person) can utilize an appropriate capillary-collection microtainer tube in order to obtain a required sample volume. Then, upon collection of the required sample volume, at step 112, the user (or other person) may close the microtainer tube with an appropriate cap or lid and bandage the user's finger at the puncture site.

Using method 100, a capillary blood sample can be obtained from a preferred off-center puncture site on the user's finger, with minimal opportunity for user error. Additionally, because the absorbent pad is integrated within the finger cuff, the first blood drop is simultaneously wiped away from the puncture site with removal of the finger cuff. In this way, the method 100 avoids an extra wiping step that is, at the very least, cumbersome to a user obtaining their own sample, and is often overlooked or skipped altogether.

While several embodiments of a finger cuff with a lancet guide and integrated absorbent pad (and a method of its use in capillary blood collection) were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A finger cuff device for use in capillary blood sample collection comprising:
a finger cuff comprising:
an open end portion sized to accommodate a user's finger,
a finger stop end portion opposite the open end portion,
an outer sidewall,
an inner sidewall, and
at least one lancet guide opening formed through the outer sidewall and inner sidewall; and
at least one integrated absorbent pad positioned on the inner sidewall of the finger cuff.

2. The finger cuff device of claim 1, wherein the at least one lancet guide opening is positioned off-center from a centerline of the finger cuff.

3. The finger cuff device of claim 2, wherein the at least one lancet guide opening is positioned proximate the finger stop end portion of the finger cuff.

4. The finger cuff device of claim 1, wherein the at least one lancet guide opening is elongated relative to a longitudinal axis of the finger cuff.

5. The finger cuff device of claim 1, wherein the at least one integrated absorbent pad is positioned on the inner sidewall in line with the at least one lancet guide.

6. The finger cuff device of claim 1, further comprising a pair of open portions formed on respective sides of the finger cuff.

7. The finger cuff device of claim 6, wherein each open portion further comprises a rounded end portion.

8. The finger cuff device of claim 1, wherein the finger cuff is formed of an elastic material.

9. A kit for capillary blood sample collection, the kit comprising:
a lancet;
a microtainer collection tube;
at least one sterile wipe;
a bandage; and
a sterile finger cuff device comprising:
a finger cuff comprising:
an open end portion sized to accommodate a user's finger,
a finger stop end portion opposite the open end portion,
an outer sidewall,
an inner sidewall, and
at least one lancet guide opening formed through the outer sidewall and inner sidewall; and
at least one integrated absorbent pad positioned on the inner sidewall of the finger cuff.

10. The kit of claim 9, wherein the at least one lancet guide opening of the finger cuff is positioned off-center from a centerline of the finger cuff.

11. The kit of claim 10, wherein the at least one lancet guide opening is positioned proximate the finger stop end portion of the finger cuff.

12. The kit of claim 9, wherein the at least one lancet guide opening of the finger cuff is elongated relative to a longitudinal axis of the finger cuff.

13. The kit of claim 9, wherein the at least one integrated absorbent pad of the sterile finger cuff device is positioned on the inner sidewall in line with the at least one lancet guide.

14. The kit of claim 9, wherein the sterile finger cuff device further comprises a pair of open portions formed on respective sides of the finger cuff.

15. The kit of claim 14, wherein each open portion of the finger cuff further comprises a rounded end portion.

16. The kit of claim 9, wherein the finger cuff is formed of an elastic material.

17. A method of capillary blood sample collection, the method comprising:
preparing a finger and desired puncture site for blood sample collection with a sterilized wipe;
placing a sterile finger cuff device over the prepared finger at the desired puncture site, the sterile finger cuff device comprising:
a finger cuff comprising:
an open end portion sized to accommodate a user's finger,
a finger stop end portion opposite the open end portion,
an outer sidewall,
an inner sidewall, and
at least one lancet guide opening formed through the outer sidewall and inner sidewall; and
at least one integrated absorbent pad positioned on the inner sidewall of the finger cuff;
pressing a lancet through the at least one lancet guide opening to puncture the finger at the desired puncture site; and
removing the sterile finger cuff device from the finger to expose the puncture site and wipe away a first blood drop from the puncture site via the at least one integrated absorbent pad of the sterile finger cuff device.

18. The method of claim 17, further comprising collecting a blood sample in a microtainer tube after the first blood drop is wiped away from the puncture site via the at least one integrated absorbent pad of the sterile finger cuff device.

19. The method of claim 18, further comprising closing the microtainer tube after collection of a desired volume of blood sample and bandaging the puncture site on the finger.

20. The method of claim 17, wherein placing the sterile finger cuff device over the prepared finger comprises placing the finger cuff on the finger such that the at least one lancet guide opening is positioned off-center of a centerline of the finger.
